SEQUENCE LISTING (23 sequences)

SEQ ID NO: 1 - protein, 4 aa, Unknown
  Description of Unknown “KDEL” motif peptide
KDEL

SEQ ID NO: 2 - DNA, 28 bp, Artificial Sequence
  Description of Artificial Sequence Synthetic primer
ctaacctattcctggtaagtggtatccg

SEQ ID NO: 3 - DNA, 42 bp, Artificial Sequence
  Description of Artificial Sequence Synthetic primer
taagcattggtaagacgtcaagcccctctgaccttgtattac

SEQ ID NO: 4 - DNA, 43 bp, Artificial Sequence
  Description of Artificial Sequence Synthetic primer
taagcattggtaagcggccgcgtgcactgatctgctagagctg

SEQ ID NO: 5 - DNA, 28 bp, Artificial Sequence
  Description of Artificial Sequence Synthetic primer
ctaatgaacacagaaggggaggtttatg

SEQ ID NO: 6 - DNA, 20 bp, Artificial Sequence
  Description of Artificial Sequence Synthetic primer
gaggggcttacaatgctttg

SEQ ID NO: 7 - DNA, 21 bp, Artificial Sequence
  Description of Artificial Sequence Synthetic primer
gggtcgttcacctagagtaag

SEQ ID NO: 8 - DNA, 24 bp, Artificial Sequence
  Description of Artificial Sequence Synthetic probe
aagagcagtagcacccagtgagtt

SEQ ID NO: 9 - DNA, 18 bp, Artificial Sequence
  Description of Artificial Sequence Synthetic primer
agatcagtgcacctacaa

SEQ ID NO: 10 - DNA, 18 bp, Artificial Sequence
  Description of Artificial Sequence Synthetic primer
caggatgcggacattgaa

SEQ ID NO: 11 - DNA, 25 bp, Artificial Sequence
  Description of Artificial Sequence Synthetic probe
agcaaactctatggaagtggaggcc

SEQ ID NO: 12 - DNA, 23 bp, Artificial Sequence
  Description of Artificial Sequence Synthetic primer
cttcttgacgagttcttctgagg

SEQ ID NO: 13 - DNA, 18 bp, Artificial Sequence
  Description of Artificial Sequence Synthetic primer
aacaacagatggctggca

SEQ ID NO: 14 - DNA, 24 bp, Artificial Sequence
  Description of Artificial Sequence Synthetic probe
tcagcctcgactgtgccttctagt

SEQ ID NO: 15 - DNA, 22 bp, Artificial Sequence
  Description of Artificial Sequence Synthetic primer
attgaggagtggcagcatatag

SEQ ID NO: 16 - DNA, 24 bp, Artificial Sequence
  Description of Artificial Sequence Synthetic primer
ggtagtctagtacctcctgtgata

SEQ ID NO: 17 - DNA, 26 bp, Artificial Sequence
  Description of Artificial Sequence Synthetic probe
tgcttccttcagcactaccctttagc

SEQ ID NO: 18 - DNA, 21 bp, Artificial Sequence
  Description of Artificial Sequence Synthetic primer
gattcagtagaccgctgttgg

SEQ ID NO: 19 - DNA, 22 bp, Artificial Sequence
  Description of Artificial Sequence Synthetic primer
gcactgggatccctacattaac

SEQ ID NO: 20 - DNA, 28 bp, Artificial Sequence
  Description of Artificial Sequence Synthetic probe
tgaaattaagagcagtagcacccagtga

SEQ ID NO: 21 - DNA, 18 bp, Artificial Sequence
  Description of Artificial Sequence Synthetic primer
tatcctcccagggaaaga

SEQ ID NO: 22 - DNA, 18 bp, Artificial Sequence
  Description of Artificial Sequence Synthetic primer
agcattgttctagaccgc

SEQ ID NO: 23 - DNA, 23 bp, Artificial Sequence
  Description of Artificial Sequence Synthetic probe
aagcccctctgaccttgtattac

(19) United States
(12) Patent Application Publication
(10) Pub. No.: US 2023/0039930 A1
(43) Pub. Date: Feb. 9, 2023

(54) METHODS AND COMPOSITIONS FOR BINDING IMMUNOGLOBULIN PROTEIN TARGETING

(71) Applicant: Sanford Burnham Prebys Medical Discovery Institute, La Jolla, CA (US)

(72) Inventor: Randal J. KAUFMAN, San Diego, CA (US)

(21) Appl. No.: 17/790,052

(22) PCT Filed: Dec. 31, 2020
(86) PCT No.: PCT/US2020/067682

§ 371 Date: Jun. 29, 2022

Related U.S. Application Data
(60) Provisional application No. 62/957,047, filed on Jan. 3, 2020.

(51) Int. Cl. A01K 67/027

(52) U.S. Cl. CPC A01K 67/0278; A01K 2267/0393; A01K 2227/105; A01K 2217/052; A01K 2217/072

(57) ABSTRACT

Models and methods related to targeting binding immunoglobulin protein (BiP) are described, where the models and methods allow identification and analysis of protein folding and misfolding.

### CROSS-REFERENCE TO RELATED APPLICATION

This application claims the benefit of U.S. Provisional Patent Application No. 62/957,047, filed on Jan. 3, 2020, which application is incorporated herein by reference.

### STATEMENT AS TO FEDERALLY SPONSORED RESEARCH

This invention was made with government support under R01 AG062190 awarded by the National Institutes of Health (NIH). The government has certain rights in the invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### BACKGROUND

Protein misfolding is a protein-specific error-prone process in all cells. In particular, ˜30% of all cellular proteins are directed into the endoplasmic reticulum (ER). Protein folding in the ER is challenging because it requires many chaperones and catalysts to assist folding and prevent aggregation in a densely packed unfavorable environment comprised of oxidizing conditions, fluctuating Ca2+ concentrations and requiring both proper disulfide bond formation and post-translational modifications (Hebert et al., In and out of the ER: protein folding, quality control, degradation, and related human diseases, Physiol Rev. 2007; 87(4):1377-408). Significantly, only proteins that achieve their appropriate 3-dimensional structures can traffic to the Golgi apparatus because of an exquisitely sensitive mechanism that identifies misfolded proteins and retains them in the ER for further productive protein folding or targets them to the degradation machinery mediated by the cytosolic 26S proteasome or macroautophagy. Protein trafficking in the ER is guided by the addition, trimming and modification of asparagine-linked core oligosaccharides in order to engage lectin-based folding machinery for proper protein triage (Hebert et al., In and out of the ER: protein folding, quality control, degradation, and related human diseases, Physiol Rev. 2007; 87(4):1377-408).

Significantly, accumulation of misfolded proteins in the ER initiates adaptive signaling through the unfolded protein response (UPR), a tripartite signal transduction pathway that transmits information about the protein folding status in the ER to the nucleus and cytosol to restore ER homeostasis (Kaufman R J, Orchestrating the unfolded protein response in health and disease, J Clin Invest. 2002; 110(10):1389-98; and Ron D et al., Signal integration in the endoplasmic reticulum unfolded protein response, Nat Rev Mol Cell Biol 2007; 8(7):519-29). If the UPR cannot resolve protein misfolding, cells may initiate cell death pathways. Stress induced by accumulation of unfolded or misfolded proteins in the ER is a salient feature of differentiated secretory cells and is observed in many human diseases including genetic diseases, cancer, diabetes, obesity, inflammation and neurodegeneration. To elucidate the fundamental etiology of these diseases it is essential to identify which proteins misfold in response to different stimuli, with a future therapeutic goal to learn how to intervene to prevent misfolding.

### SUMMARY OF THE DISCLOSURE

The present disclosure provides models and methods for epitope-tagging of the endogenous BiP/GRp78/Hspa5 locus. The disclosed models and methods allow direct analysis of the BiP interactome and protein folding and misfolding in vivo. In some embodiments, disclosed herein are models of protein folding comprising a transgenic animal with a transgene comprising an epitope tag in the Hspa5 gene. In some embodiments, the transgenic animal comprises a mammal. In some embodiments, the transgenic animal is selected from the group consisting of a mouse, a rat, and a monkey. In some embodiments, the transgenic animal is a mouse. In some embodiments, the transgenic animal is produced via homologous recombination. In some embodiments, the epitope tag is selected from the group consisting of GST, streptavidin, poly(His), FLAG-tag, V5-tag, Myc-tag, HA-tag, Spot-tag, T7-tag, and NE-tag. In some embodiments, the epitope tag comprises a FLAG-tag. In some embodiments, the FLAG-tag comprises at least three FLAG sequences.

In some embodiments, disclosed herein are BiP-FLAG mice characterized by FLAG-tagged BiP-client complexes. In some embodiments, the FLAG-tagged BiP-client complexes comprise at least three FLAG sequences.

### BRIEF DESCRIPTION OF THE DRAWINGS

The patent application contains at least one drawing executed in color. Copies of this patent or patent application with color drawing(s) will be provided by the Office upon request and payment of the necessary fee.

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which.

FIG. 1A depicts the targeting strategy for the generation of BiP-FLAG-Het mice by homologous recombination. The replacement vector contains a Neo-cassette (yellow) flanked by FRT sites (green) which, together with targeted WT exon 9-pA cassette, are flanked by LoxP sites (red) followed by duplicated exon 9 containing a 3xFLAG sequence(red) upstream from the KDEL motif. Using 5′ and 3′ homology arms the vector was used to target the Hspa5 locus of murine ES cells. The Neo-cassette was removed by Flp-mediated recombination. After Cre mediated recombination, exon 9-FLAG is expressed under control of the endogenous Hspa5 regulatory elements. Blue boxes denote exons. Lo5WT, Lo3WT and GoConK represent qPCR probes for the specified locus.

FIG. 1B depicts the copy number quantification. qPCR was performed to analyze BiP-FLAG-Het mice. qPCR reactions display genotyping from WT/WT-Flp/Flp, WT/conKI-WT/Flp (mice number, 2093_044_A048F), and WT/conKIWT-WT/Flp (mice number, 2093_044_A047F) mice. Primers are indicated in panel A. Lo5WT and LoWT3 identify the WT allele of Hspa5. goConK identifies the knock-in allele of Hspa5.

FIG. 1C depicts targeted allele expression. Total RNA was extracted from BiP-FLAG-Het mice liver infected with AAV8-TBG-Cre. mRNA expression of Hspa5 was measured by qRT-PCR.

FIG. 2 depicts the BiP-FLAG allele is induced by ER stress in primary hepatocytes. Hepatocytes were isolated from a 6-wk old female BiP-FLAG-Het mouse, plated onto 24 well plates and infected with the indicated adenoviruses at 4 hours after plating. After 48 hours, cells were treated with Tm 0.5 μg/ml or vehicle for 20 h and then harvested for Western blot analysis.

FIG. 3 depicts BiP-FLAG is activated by Ad-Cre infection in BiP-FLAG primary fibroblasts. Skin fibroblasts were isolated from a 6-wk old female BiP-FLAG-Het mouse and plated onto 24 well plates, infected with the indicated adenoviruses after the first passage and harvested for Western blot analysis.

FIG. 4 depicts BiP-FLAG is induced in BiP-FLAG primary fibroblasts in response to ER stress. Skin fibroblasts were isolated from a 6-wk old female BiP-FLAG-Het mouse, plated onto 24 well plates, infected with the indicated adenoviruses after the first passage. After Ad-infection, cells were treated with Tm 0.5 μg/ml, CST 20 μM or vehicle for 22 hours and harvested for Western blot analysis.

FIGS. 5A-B depicts BiP-FLAG is localized to the ER lumen. FIG. 5A depicts that hepatocytes were isolated from a 6-wk old female BiP-FLAG-Het mouse, plated onto 6 well plates and infected with the indicated adenoviruses at 4 hours after plating. After 4 days, cells were fixed with formalin and stained with anti-FLAG antibody, anti-PDIA6 antibody and DAPI. Images were captured by a 63 oil lens from each group by confocal microscopy. Scale bar, 20 μm. FIG. 5B depicts that fibroblasts were isolated from a 6-wk old female BiP-FLAG-Het mouse, plated onto 6 well plates and infected with the indicated adenoviruses at 7 days after plating. At 24 hours after Ad-infection, cells were fixed with formalin, stained with antibodies for FLAG or PDIA6 and for DAPI. Images were captured by a 63 oil lens from each group by confocal microscopy. Scale bar, 20 μm.

FIGS. 6A-C depicts AAV8-TBG-Cre-treatment of BiP-FLAG-Het mice does not alter plasma or hepatic lipid content. Plasma cholesterol (FIG. 6A) and triglyceride (FIG. 6B) levels and hepatic triglyceride (FIG. 6C) content of AAV8-TBG-Cre-treated Hspa5 wildtype (WT) and BiP-FLAG-Het (Het) mice at 17 h after injection with vehicle (PBS) or Tm were measured. Each data point represents one individual mouse.

FIG. 7 depicts BiP-FLAG induction in hepatocytes by ER stress in vivo. After infection with AAV8-TBG-Cre or control virus for 10 days, mice were treated with Tm (1 mg/Kg) or vehicle (saline). After 17 h, liver tissues were collected immediately after sacrifice and for lysis in RIPA-buffer. Each lane in the Western blot represents an individual mouse.

FIG. 8 depicts hepatocyte-specific BiP-FLAG knock-in does not alter expression of key unfolded protein response (UPR) genes in the liver. Total RNAs were isolated from liver samples collected as described in FIG. 7 and subjected to qRT-PCR analysis to measure the mRNA levels for the indicated genes normalized to 18S rRNA.

FIG. 9 depicts BiP-FLAG is efficiently pulled down from AAV8-TBG-Cre/BiP-FLAG-Het liver lysates using murine anti-Flag (M2) agarose. Liver lysates prepared as described in FIG. 7 were subjected to immuno-precipitation (IP) using anti-FLAG (m2)-coupled to agarose. Western blotting was performed using a rabbit anti-BiP monoclonal antibody (3177, CST) as a primary antibody.

FIG. 10 depicts body weight of BiP-FLAG and wild type (WT) mice. Body weights were measured at 6-8 weeks of age from WT and BiP-FLAG knock-in mice. Wt, males N=6, females N=4. BiP-FLAG, males N=2, females N=5.

FIGS. 11A-B depicts AAV infection efficiency in primary hepatocytes and fibroblasts. FIG. 11A depicts that hepatocytes were isolated from a 6-wk old female BiP-FLAG-Het mouse, plated onto 6 well plates and infected with the indicated adenoviruses at 4 hours after plating. After 24 hours, cells were fixed with formalin and stained with anti-FLAG antibody and DAPI. FIG. 11B depicts that fibroblasts were isolated from a 6-wk old female BiP-FLAG-Het mouse, plated onto 6 well plates and infected with the indicated adenoviruses at 7 days after plating. At 24 hours after Ad-infection, cells were fixed with formalin and staining with anti-FLAG antibody and DAPI. Four Images were randomly captured from each group by Zeiss 710 confocal microscopy. Scale bar, 50 μm. Quantification was performed by Image J. Quantification of the ratio of FLAG-positive cells to DAPI positive cells is shown in each graph (right).

FIGS. 12A-B depicts liver histology of AAV-TBG-Cre infected BiP-FLAG-Het mice. FIG. 12A depicts morphology of hepatocytes based on H&E stained liver sections of experimental mice. FIG. 12B depicts pathohistological analysis and morphology of hepatocytes based on Oil Red O stained liver sections of experimental mice. Experiments were performed with 2 mice in each group. Stained sections were scanned by Aperio, Leica Biosystems. The scale bar is 200 μm.

FIGS. 13A-B depicts that proteomics analysis of BiP-FLAG complexes isolated from livers of vehicle- and tunicamycin (Tm)-treated BiP-FLAG heterozygous mice demonstrates the feasibility to use BiP-FLAG-expressing mice to isolate BiP-interactome and ER misfolded proteins. Mass Spec analysis was carried out using BiP-FLAG complexes isolated from the livers of vehicle- and Tm-treated BiP-FLAG heterozygous and wild type mice described above. FIG. 13A. A summary of proteins that exhibited augmented interaction with BiP-FLAG in response to Tm-treatment; FIG. 13B. Effect of Tm-treatment on interactions of ER proteins with BiP-FLAG. Note, Tm treatment reduced BiP interactions with UPR sensors PERK (Eif2ak3) and IRE1α (Ern1) (gray arrows), indicating their release from BiP due to UPR activation, while promoting BiP interactions with chaperone proteins such as Grp94 (Hsp90b1), GRP170 (Hyou1), and P58 (Dnajc3) (black arrows).

### DETAILED DESCRIPTION

The present disclosure is based on the finding that successful epitope-tagging of the endogenous murine BiP/GRP78/Hspa5 locus allows direct analysis of the BiP interactome and protein misfolding in vivo. BiP/GRP78, encoded by the Hspa5 gene, is the major HSP70 family member in the endoplasmic reticulum (ER) lumen, and controls ER protein folding. BiP's essential functions in facilitating proper protein folding are mainly mediated through its dynamic interaction with unfolded or misfolded client proteins, and by serving as a negative regulator of the Unfolded Protein Response. A mechanistic understanding of the dynamics of BiP interaction with its protein partners is essential to understand ER biology, and therefore, disclosed herein are tractable models to study misfolded protein interaction with BiP. In some embodiments, disclosed herein are tractable models created using homologous recombination to insert a 3xFLAG epitope tag into the endogenous murine Hspa5 gene, just upstream from the essential KDEL signal necessary for ER localization of BiP. As disclosed herein, tagging BiP in this way did not alter Hspa5 expression under basal or ER-stress induced conditions in hepatocytes ex vivo or in fibroblasts. Furthermore, the tag did not alter the cellular localization of BiP or its functionality. As disclosed herein, all of these findings in primary tissue culture were also confirmed in vivo in livers of heterozygous mice with one WT and one FLAG-tagged Hspa5 allele. Hepatocyte-specific BiP-FLAG modification did not alter liver function or UPR signaling. Importantly, immunoprecipitation with anti-FLAG antibody completely pulled down FLAG-tagged BiP from lysates of BiP-FLAG expressing livers. In summary, disclosed herein is a novel model that can be used to investigate the BiP interactome in vivo under physiological and pathophysiological conditions in a cell type-specific manner. This model provides for the first time an unbiased approach to identify unfolded and misfolded BiP-client proteins, and to provide new information on the role of BiP in many essential ER processes.

The characterization of protein misfolding in vivo under different physiological conditions is limited due to the absence of conformation-specific antibodies, which are available for some viral glycoproteins, but are mostly lacking for endogenous cellular proteins. In addition, there is a need for an unbiased approach to identify the full spectrum of unfolded and misfolded proteins in the ER, in order to uncover the extent of misfolding of different protein species during disease progression, as well as the impact of different stimuli that can exacerbate ER protein misfolding. The most reliable surrogate for the misfolding of ER client proteins is their interaction with the “Binding Protein” known as BiP (encoded by HSPA5) which is a heat-shock protein 70 ER chaperone exhibiting peptide-dependent ATPase. BiP was originally characterized as a protein that binds immunoglobulin heavy chains to maintain them in a folding-competent state prior to their oligomerization with light chains (Haas I G et al., Immunoglobulin heavy chain binding protein, Nature. 1983; 306(5941):387-9). It was also recognized that glucose-deprivation induces a set of genes encoding glucose-regulated proteins, the most abundant being the ER protein GRP78, which is identical to BiP (Munro S et al., An Hsp70-like protein in the ER: identity with the 78 kd glucose-regulated protein and immunoglobulin heavy chain binding protein, Cell. 1986; 46(2):291-300). Further analysis demonstrated that BiP expression is induced by protein misfolding in the ER through activation of the UPR.

Intriguingly, increased BiP levels feed-back to negatively regulate further UPR activation. One hypothesis posits that BiP binding to the UPR sensors IRE1, ATF6 and PERK inhibits their signaling (Bertolotti A et al., Dynamic interaction of BiP and ER stress transducers in the unfolded-protein response, Nat Cell Biol. 2000; 2(6):326-32), although there is no direct evidence to support this notion in physiological settings in vivo. Early studies to analyze protein misfolding demonstrated that only misfolded proteins that bind BiP activate the UPR and those that do not bind BiP do not activate the UPR (Dorner et al., The relationship of N-linked glycosylation and heavy chain-binding protein association with the secretion of glycoproteins, J Cell Biol. 1987; 105(6 Pt 1):2665-74; Kozutsumi et al., The presence of malfolded proteins in the endoplasmic reticulum signals the induction of glucose-regulated proteins, Nature. 1988; 332(6163):462-4; Dorner et al., Increased synthesis of secreted proteins induces expression of glucose-regulated proteins in butyrate-treated Chinese hamster ovary cells, J Biol Chem. 1989; 264(34):20602-7; Dorner et al., Overexpression of GRP 78 mitigates stress induction of glucose regulated proteins and blocks secretion of selective proteins in Chinese hamster ovary cells, EMBO J. 1992; 11(4):1563-71; Ng et al., Analysis in vivo of GRP78-BiP/substrate interactions and their role in induction of the GRP78-BiP gene, Mol Biol Cell. 1992; 3(2):143-55; Scheuner et al., Control of mRNA translation preserves endoplasmic reticulum function in beta cells and maintains glucose homeostasis, Nat Med. 2005; 11(7):757-64; and Hidvegi et al., Accumulation of mutant alpha1-antitrypsin Z in the endoplasmic reticulum activates caspases-4 and -12, NFkappaB, and BAP31 but not the unfolded protein response, J Biol Chem. 2005; 280(47):39002-15). Unfortunately, however, there are no BiP antibodies currently available that can efficiently recognize BiP-client protein complexes in the absence of chemical crosslinkers, thus limiting the ability to study protein misfolding in the ER. As BiP provides many essential ER functions (including regulating Sec61 for co-translational and post-translational translocation into the ER, protein folding and degradation, maintenance of ER Ca2+ stores, repressing UPR signaling, etc.), characterizing BiP interactions in vivo is essential to understand all these processes, and will provide significant insight into the role of ER protein misfolding in disease pathogenesis.

BiP immunoprecipitation (IP) from whole tissue lysates has the limitation that BiP is ubiquitously expressed; thus, IP recovers BiP and its partner proteins from multiple cell types. The models and methods disclosed herein overcome this challenge and provide the ability to follow cell type-specific BiP interactions at different stages of disease progression. In addition, the models and methods disclosed herein avoid BiP overexpression, because this increases non-physiological BiP interactions (Dorner et al., Overexpression of GRP78 mitigates stress induction of glucose regulated proteins and blocks secretion of selective proteins in Chinese hamster ovary cells, EMBO J. 1992; 11(4):1563-71). In some embodiments, disclosed herein are models constructed using homologous recombination to generate a conditional allele in mice with insertion of a 3xFLAG tag into the C- terminus of the endogenous BiP (Hspa5) coding sequence, just upstream from the KDEL ER localization signal. The engineered allele is designed such that upon cell type-specific Cre-induced deletion, expression of BiP-3xFLAG from the endogenous locus will permit endogenous BiP expression with the ability to identify BiP-interactors by anti-FLAG IP.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively, unless the context specifically refers to a disjunctive use.

The term “about” or “approximately” may mean within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Alternatively, particularly with respect to biological systems or processes, the term may mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the present disclosure, and vice versa. Furthermore, compositions of the present disclosure may be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Reference in the specification to a “cell” may refer to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell may be a synthetically made, sometimes termed an artificial cell).

Reference in the specification to “nucleotide,” as used herein, refers to a base-sugar-phosphate combination. A nucleotide may comprise a synthetic nucleotide. A nucleotide may comprise a synthetic nucleotide analog. Nucleotides may be monomeric units of a nucleic acid sequence (e.g. deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [aS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled by well-known techniques. Labeling may also be carried out with quantum dots. Detectable labels may include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,NcN′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides may include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAN1RA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, TR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides may also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-cICTP, biotin-14-dCTP), and biotin-dUTP (e.g. biotin-11-dUTP, biotin-1.6-dUTP, biotin-20-dUTP).

Terms such as “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure, and may perform any function, known or unknown. A polynucleotide may comprise one or more analogs (e.g. altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, eDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

Reference in the specification to “conjugated” may be used to designated chemically bonded i.e., attached by chemical bonds. A conjugate is a molecule, example a peptide that is chemically (for example covalently) linked to a biomolecule or molecule of interest, for example, a nucleic acid, that is conjugated to another molecule.

Reference in the specification to “operably linked” refers to a functional relationship between two or more nucleic acid segments. Typically, it refers to the functional relationship of a transcriptional regulatory sequence to a transcribed sequence.

“Polyadenylation sequence” (also referred to as a “poly A⁺ site” or “poly A⁺ sequence”) refers to a DNA sequence which directs both the termination and polyadenylation of the nascent RNA transcript. Efficient polyadenylation of the recombinant transcript is desirable, as transcripts lacking a poly A⁺ tail are typically unstable and rapidly degraded. The poly A⁺ signal utilized in an expression vector may be “heterologous” or “endogenous”. An endogenous poly A⁺ signal is one that is found naturally at the 3′ end of the coding region of a given gene in the genome. A heterologous poly A⁺ signal is one which is isolated from one gene and placed 3′ of another gene, e.g., coding sequence for a protein. A commonly used heterologous poly A⁺ signal is the SV40 poly A⁺ signal. The SV40 poly A⁺ signal is contained on a 237 bp BamHI/BclI restriction fragment and directs both termination and polyadenylation; numerous vectors contain the SV40 poly A⁺ signal. Another commonly used heterologous poly A⁺ signal is derived from the bovine growth hormone (BGH) gene; the BGH poly A⁺ signal is also available on a number of commercially available vectors. The poly A⁺ signal from the Herpes simplex virus thymidine kinase (HSV tk) gene is also used as a poly A⁺ signal on a number of commercial expression vectors. The polyadenylation signal facilitates the transportation of the RNA from within the cell nucleus into the cytosol as well as increases cellular half-life of such an RNA. The polyadenylation signal is present at the 3′ -end of an mRNA.

Reference in the specification to “exon” refers to a nucleic acid sequence found in genomic DNA that is bioinformatically predicted and/or experimentally confirmed to contribute contiguous sequence to a mature mRNA transcript.

Reference in the specification to “intron” refers to a sequence present in genomic DNA that is bioinformatically predicted and/or experimentally confirmed to not encode part of or all of an expressed protein, and which, in endogenous conditions, is transcribed into RNA (e.g. pre-mRNA) molecules, but which is spliced out of the endogenous RNA (e.g. the pre-mRNA) before the RNA is translated into a protein.

Reference in the specification to “complement,” “complements,” “complementary,” and “complementarity,” as used herein, can refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/embossneedle/nucleotide.html), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.ukaools/psa/emboss_water/nucleotide.html, optionally with default settings). Optimal alignment can be assessed using any suitable parameters of a chosen algorithm, including default parameters. Complementarity may be perfect or substantial/sufficient. Perfect complementarity between two nucleic acids may mean that the two nucleic acids may form a duplex in which every base in the duplex is bonded to a complementary base by Watson-Crick pairing. Substantial or sufficient complementary may mean that, a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions may be predicted by using the sequences and standard mathematical calculations to predict the melting temperature (Tₘ) of hybridized strands, or by empirical determination of Tₘ by using routine methods.

The term “knockout” (“KO”) or “knocking out” as used herein refers to a deletion, deactivation, or ablation of a gene in a cell, or in an organism, such as, in a pig or other animal or any cells in the pig or other animal. KO, as used herein, may also refer to a method of performing, or having performed, a deletion, deactivation or ablation of a gene or portion thereof, such that the protein encoded by the gene is no longer formed.

The term “knockin” (“KI”) or “knocking in” as used herein refers to an addition, replacement, or mutation of nucleotide(s) of a gene in a pig or other animal or any cells in the pig or other animal. KI, as used herein, may also refer to a method of performing, or having performed, an addition, replacement, or mutation of nucleotide(s) of a gene or portion thereof.

The terms “peptide,” “polypeptide,” and “protein” are used interchangeably herein to refer. to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

Reference in the specification to “derivative,” “variant,” and “fragment,” may be with regards to a polypeptide, can indicate a polypeptide related to a wild type polypeptide, for example either by amino acid sequence, structure (e.g., secondary and/or tertiary), activity (e.g., enzymatic activity) and/or function. Derivatives, variants and fragments of a polypeptide may comprise one or more amino acid variations (e.g., mutations, insertions, and deletions), truncations, modifications, or combinations thereof compared to a wild type polypeptide.

Reference in the specification to “percent (%) identity,” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps may be introduced in one or both of the candidate and reference sequences for optimal alignment and non-homologous sequences may be disregarded for comparison purposes). Alignment, for purposes of determining percent identity, may be achieved in various ways that are within the skill in the art, for instance, using publicly available computer software such as BLAST, ALIGN, or Megalign (DNASTAR) software. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

Reference in the specification to “nucleic acid editing moiety,” can indicate a moiety, which may induce one or more gene edits in a polynucleotide sequence. The polynucleotide sequence may be in a host cell. Alternatively, the polynucleotide sequence may not be in a host cell. Gene editing using the nucleic acid editing moiety may comprise introducing one or more heterologous polynucleic acids (for example, genes, or fragments thereof) in a cell, or deleting one or more endogenous polynucleic acids (for example, genes, or fragments thereof) from the cell. In some cases, gene editing using the nucleic acid editing moiety may comprise substituting any one or more polynucleic acids (for example, genes, or fragments thereof) thereof. In some cases, gene editing using the nucleic acid editing moiety may comprise a combination of any of the above, either simultaneously or sequentially. In some cases, the one or more polynucleic acids may be a DNA. In some cases, the one or more polynucleic acids may be genomic DNA. In some cases, the any one or more genes or nucleic acid portions thereof may be added to or deleted from the chromosomal DNA of a cell by the nucleic acid editing moiety. In some cases, the one or more polynucleic acids may be genomic DNA. In some cases, one or more polynucleic acids may be added to or deleted from the chromosomal DNA of a cell by the nucleic acid editing moiety, that is not part of a gene. In some cases, the one or more polynucleic acids may be contained in exosomes. In some cases, one or more polynucleic acids may be in mitochondria or any other cell organelle. In some cases, the any one or more genes or nucleic acid portions thereof may be added to or deleted from the episomal DNA or epichromosomal DNA of the cell by the nucleic acid editing moiety. In some cases, one or more polynucleic acids may be RNA. In some cases, one or more exogenous polynucleic acids may be added into the genomic DNA, via integration of the exogenous polynucleic acids into the genomic DNA. Integration of any one or more genes into the genome of a cell may be done using any suitable method. Non-limiting examples of suitable methods for the genomic integration and/or genomic replacement strategies disclosed and described herein include CRISPR-mediated genetic modification using Cas9, Cas12a (Cpf1), or other CRISPR endonucleases, Argonaute endonucleases, transcription activator-like (TAL) effector and nucleases (TALEN), zinc finger nucleases (ZFN), expression vectors, transposon systems (e.g., PiggyBac transposase), or any combination thereof. Designer zinc fingers, transcription activator-like effectors (TALEs), or homing meganucleases are available for producing targeted genome perturbations.

Targeted genome editing is possible via CRISPR-mediated genetic modification using a Cas or Cas-like endonuclease. CRISPR (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli, and associated genes. Similar interspersed SSRs may be identified in Haloferax mediterranei, Streptococcus pyogenes, Anabaena, and Mycobacterium tuberculosis. The CRISPR loci typically differ from other SSRs by the structure of the repeats, which have been termed short regularly spaced repeats (SRSRs). The repeats are short elements that occur in clusters that are regularly spaced by unique intervening sequences with a substantially constant length. Although the repeat sequences are highly conserved between strains, the number of interspersed repeats and the sequences of the spacer regions typically differ from strain to strain. CRISPR loci have been identified in more than 40 prokaryotes including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

Cas9 gene may be found in several diverse bacterial genomes, typically in the same locus with cas1, cas2, and cas4 genes and a CRISPR cassette. Furthermore, the Cas9 protein contains a readily identifiable C-terminal region that is homologous to the transposon ORF-B and includes an active RuvC-like nuclease, an arginine-rich region. A Cas 9 protein may be from an organism from a genus comprising, Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, or Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, or Acidaminococcus.

The nucleic acid editing moiety may comprise a nucleic acid cleavage moiety. The nucleic acid cleavage moiety may introduce a break or a cleavage in a nucleic acid site molecule. The nucleic acid cleavage moiety may be capable of recognizing a specific cleavage recognition site, for example, when in proximity to the cleavage recognition site on a target polynucleotide sequence. In some cases, the nucleic acid cleavage moiety may be directed by a second molecule (such as a nucleic acid, e.g. sequence specific guide RNA for Cas9) to a specific cleavage site on a polynucleic acid, for introducing a break or cleavage on the polynucleic acid. The nucleic acid cleavage moiety may initiate an introduction, deletion or substitution of the nucleic acid in the genomic DNA. In some cases, the nucleic acid cleavage moiety is a nuclease, or a functional fragment thereof. In some cases, the nucleic acid cleavage moiety may comprise an endonuclease, an exonuclease, a DNase, an RNase, a strand-specific nuclease, or a more specialized nuclease, (for example, a CRISPR associated protein 9, Cas 9), or any fragment thereof. In some cases, the nucleic acid cleavage moiety may be nickase.

In some cases, the nuclease is an AAV Rep protein, Rep68/78.

In some cases, a nucleic acid editing moiety may comprise a viral machinery or a fragment thereof that is capable of incorporating a viral gene into a host cell. For example, a nucleic acid editing moiety may refer to a viral integrase system, such as a lentiviral integrase system. Integrase is a retroviral enzyme that catalyzes integration of DNA into the genome of a mammalian cell, a useful step of retrovirus replication in the retroviral infection process. The process of integration can be divided into two sequential reactions. The first one, named 3′-processing, corresponds to a specific endonucleolytic reaction which prepares the viral DNA extremities to be competent for the subsequent covalent insertion, named strand transfer, into the host cell genome by a trans-esterification reaction. In some cases, a nucleic acid editing moiety may additionally refer to a transposon/transposase or a retrotransposase system or a component thereof, for integration of a piece of DNA into the genome. However, inserting exogenous DNA into specific genomic sequences is preferred over random and semi-random integration throughout the target cell's genome, such as with some retroviral vectors and transposons/transposases. The random and semi-random integration procedures may result in outcomes such as positional-effect variegation, transgene silencing, and, in some cases, insertional mutagenesis caused by transcriptional deregulation or physical disruption of endogenous target-cell genes.

Reference in the specification to antisense oligomeric nucleic acids or antisense oligonucleotides or ASOs refers to antisense RNA, that can be synthetic single-stranded deoxyribonucleotide analogs, usually 15-30 bp in length. Their sequence (3′ to 5′) is antisense and complementary to the sense sequence of the target nucleotide sequence. Unmodified oligonucleotides after quick degradation by circulating nucleases are excreted by the kidney; unmodified oligonucleotides are generally too unstable for therapeutic use. Therefore, chemical modification strategies have been developed to overcome this and other obstacles in ASO therapy program. Commonly used modification in these ASOs is 2′ ribose modifications that include 2′-O-methoxy (OMe), 2′-O-methoxy-ethyl (MOE), and locked nucleic acid (LNA). 2′-OMe modifications are commonly used in a ‘gapmer’ design, which is a chimeric oligo comprising a DNA sequence core with flanking 2′-MOE nucleotides that enhances the nuclease resistance, in addition to lowering toxicity and increasing hybridization affinities. Sequence specific “small inhibiting RNA (siRNA)” or “iRNA” relates to small RNA sequences that bind to a target nucleic acid molecule, which can expression of a gene expression product. Introduction of double-stranded RNA (dsRNA) also called interfering RNA (RNAi), or hairpin RNA is an effective trigger for the induction of gene silencing in a large number of eukaryotic organisms, including animals, fungi, and plants. Both the qualitative level of dsRNA-mediated gene silencing (i.e., the level of gene silencing within an organism) and the quantitative level (i.e., the number of organisms showing a significant level of gene silencing within a population) have proven superior to the more conventional antisense RNA or sense RNA mediated gene silencing methods.

Another method of inhibiting gene expression comprises targeting a nucleic acid molecule to an anti-sense transcript and sense strand transcript, wherein the nucleic acid molecule targeting the anti-sense transcript is complementary to the anti-sense strand and the nucleic acid molecule targeting the sense transcript is complementary to the sense strand; and, binding of the nucleic acid to the anti-sense and sense transcript, thereby, inhibiting gene expression. The nucleic acid molecule is a RNA molecule and the nucleic acid molecules targeting the anti-sense and sense transcripts bind said transcripts in convergent, divergent orientations with respect to each other and/or are overlapping. Method for gene suppression in eukaryotes by transformation with a recombinant construct containing an antisense and/or sense nucleotide sequence for the gene(s) to be suppressed is known in the art.

In some embodiments, the models and methods disclosed herein make use of a vector. A large number of vector and promoter systems are well known in the art. Construction of expression vectors having a promoter that is inducible by a regulator is known to one of skill in the art. Exemplary inducible promoter may be a doxycycline or a tetracycline inducible promoter. Tetracycline regulated promoters may be both tetracycline inducible or tetracycline repressible, called the tet-on and tet-off systems. The tet regulated systems rely on two components, i.e., a tetracycline-controlled regulator (also referred to as transactivator) (tTA or rtTA) and a tTA/rtTA-dependent promoter that controls expression of a downstream cDNA, in a tetracycline-dependent manner. tTA is a fusion protein containing the repressor of the Tn10 tetracycline-resistance operon of Escherichia coli and a carboxyl-terminal portion of protein 16 of herpes simplex virus (VP16). The tTA-dependent promoter consists of a minimal RNA polymerase II promoter fused to tet operator (tetO) sequences (an array of seven cognate operator sequences). This fusion converts the tet repressor into a strong transcriptional activator in eukaryotic cells. In the absence of tetracycline or its derivatives (such as doxycycline), tTA binds to the tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. In contrast, in the tet-ON system, a mutant form of tTA, termed rtTA, has been isolated using random mutagenesis. In contrast to tTA, rtTA is not functional in the absence of doxycycline but requires the presence of the ligand for transactivation. A Tamoxifen inducible system may comprise a reversible switch, that can provide reversible control over the transcription of a gene or genes that are regulated by the system. The tamoxifen/estrogen receptor regulatable system can allow spatiotemporal control of gene expression, especially when combined with the Cre/Lox recombinase system, where the Cre recombinase is fused to a mutant form of the ligand-binding domain of the human estrogen receptor resulting in a tamoxifen-dependent Cre recombinase.

The present disclosure provides models and methods for targeting binding immunoglobulin protein (BiP), which allow an unbiased approach to identify unfolded and misfolded BiP-client proteins, and provide new information on the role of BiP in many essential ER processes. In some embodiments, disclosed herein are models demonstrating successful targeting of the endogenous Hspa5 locus in mice. Phenotypic characterization of the models disclosed herein identified no defect in hepatocyte function, ER function or BiP-FLAG localization to the ER. The models disclosed herein provide information on BiP client specificity, function, and role in protein folding. The models disclosed herein also address questions of protein misfolding and binding in healthy versus diseased cells; whether inducers of ER stress generate similar protein misfolding consequences or are there differences depending on the degree or type of ER stress or cell type; which proteins exist in different complexes that contain BiP; what are the kinetics of misfolded protein interaction and release from BiP; and how do BiP interactions impact general ER processes. The models disclosed herein enable for the first-time delineation of folding pathways for any specific protein in vivo.

### EXAMPLES

### Example 1

### Generation of BiP-3xFLAG Mice

This example illustrates generation of an exemplary mouse model for targeting binding immunoglobulin protein (BiP). A conditional knock-in mouse model was generated by modifying of the Hspa5 locus. This was achieved by floxing a targeted WT exon 9-pA cassette upstream of the knock-in exon 9 where a 3xFLAG sequence was introduced immediately prior to the KDEL ER retention signal. Additionally, a FRT-flanked neomycin cassette was introduced into the foxed region. The genetic modification was introduced into Bruce4 C57BL/6 ES cells (Kontgen et al., Targeted disruption of the MHC class II Aa gene in C57BL/6 mice, Int. Immunol. 1993; 5(8):957-64) via gene targeting. Correctly targeted ES cell clones were identified and then injected into goGermline blastocysts (Koentgen et al., Exclusive transmission of the embryonic stem cell-derived genome through the mouse germline, Genesis 2016; 54(6):326-33; and Zhou et al., The testicular soma of Tsc22d3 knockout mice supports spermatogenesis and germline transmission from spermatogonial stem cell lines upon transplantation, Genesis 2019; 57(6):e23295). Male goGermline mice were bred to C57BL/6 females to establish heterozygous germline offspring on a C57BL/6 background.

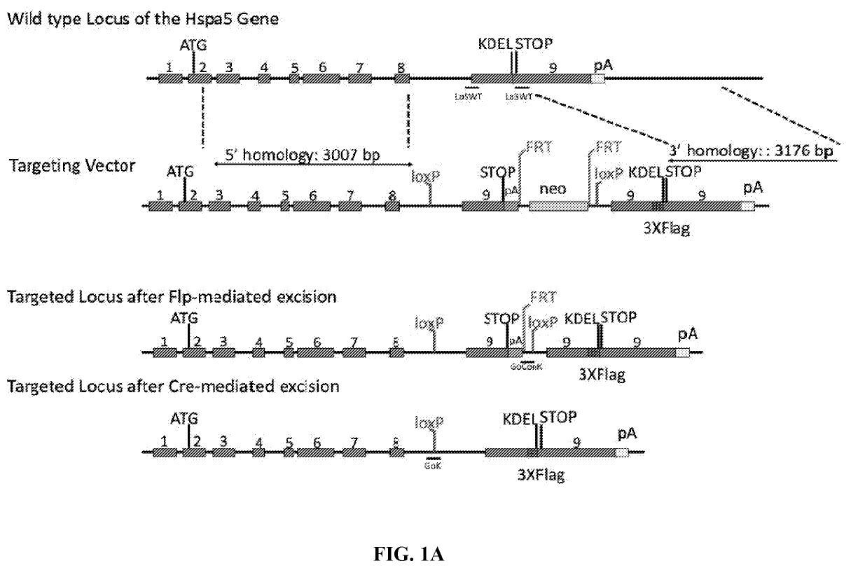

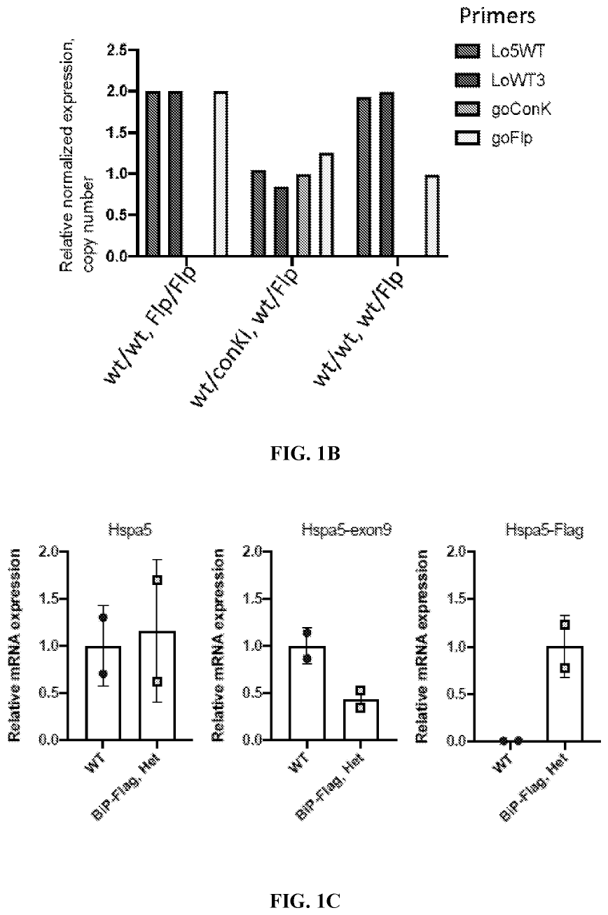

The vector was constructed as follows. A replacement vector targeting Hspa5 exon 9 coding sequence region (CDS region) was generated by assembly of 4(ABCD) fragments using sequential cloning. The first fragment which encompassed the 3 kb 5′-homology arm was generated by PCR amplified from C57BL/6 genomic DNA using primers P2093_41 and P2093_51. The second and third fragments which comprise loxP-exon9-BGHpA and exon 9-3xFlag were synthesized by Genewiz, respectively. The fourth fragment comprising the 3.2 kb 3′-homology arm was generated by PCR amplified from C57BL/6 genomic DNA using primers P2093_44 and P2093_54. Synthesized fragments and PCR primers used to amplify the fragments included all the restriction enzyme sites required to join them together and to ligate them into the Surf2 vector backbone (Ozgene). The final targeting vector 2093_Teak_ABCD contained a FRT-flanked neomycin selection (neo) cassette, an exon 9 coding sequence sequentially with an inserted bovine growth hormone (BGH) polyA tail, an additional exon 9 coding sequence sequentially with a 3xFLAG tag cassette right before the KEDL sequence, 5′ and 3′-loxP site (FIG. 1A). Sequence information of the primers appears in Table 1, below.

TABLE 1
Primer Sequence Information

| Name | Sequence | Used for |
|---|---|---|
| P2093_41 | CTAACCTATTCCTGGTAAGTGGTATCCG | Targeting vector construction |
| P2093_51 | TAAGCATTGGTAAGACGTCAAGCCCCTCTGAGTATTAC | Targeting vector construction |
| P2093_44 | TAAGCATTGGTAAGCGGCCGCGTGCACTGATGCTAGAGCTG | Targeting vector construction |
| P2093_54 | CTAATGAACACAGAAGGGGAGGTTTATG | Targeting vector construction |
| 2093_Lo5WT_F | GAGGGGCTTACAATGCTTTG | 5′ end Hspa5 wt allele validation |
| 2093_Lo5WT_R | GGGTCGTTCACCTAGAGTAAG | 5′ end Hspa5 wt allele validation |
| 2093_Lo5WT_Probe | AAGAGCAGTAGCACCCAGTGAGTT | 5′ end Hspa5 wt allele validation |
| 2093_LoWT3_F | AGATCAGTGCACCTACAA | 3′ end Hspa5 wt allele validation |
| 2093_LoWT3_R | CAGGATGCGGACATTGAA | 3′ end Hspa5 wt allele validation |
| 2093_LowT3_Probe | AGCAAACTCTATGGAAGTGGAGGCC | 3′ end Hspa5 wt allele validation |
| 1638_goNoz_F | CTTCTTGACGAGTTCTTCTAGG | Gain of Neo validation |
| 1638_goNoz_R | AACAACAGATGGCTGGCA | Gain of Neo validation |
| 1628_goNoz_Probe | TCAGCCTCGACTGTGCCTTCTAGT | Gain of Neo validation |
| 1638_goFlpOz_F | ATTGAGGAGTGGCAGCATATAG | Gain of Flp validation |
| 1638_goFlpOz_R | GGTAGTCTAGTACCTCCTGTGATA | Gain of Flp validation |
| 1638_goFlpOz_Probe | TGCTTCCTTCAGCACTACCCTTTAGC | Gain of Flp validation |
| 2093_GoConK_F | GATTCAGTAGACCGCTGTTGG | Gain of 2093_Teak conKI allele |
| | | validation |
| 2093_GoConK_R | GCACTGGGATCCCTACATTAAC | Gain of 2093_Teak conKI allele |
| | | validation |
| 2093_GoConK_Probe | TGAAATTAAGAGCAGTAGCACCCAGTGA | Gain of 2093_Teak conKI allele |
| | | validation |
| 2093_GoK_F | TATCCTCCCAGGGAAAGA | Gain of 2093_Teak conKI allele |
| | | validation |
| 2093_GoK_R | AGCATTGTTCTAGACCGC | Gain of 2093_Teak conKI allele |
| | | validation |
| 2093_GoK_Probe | AAGCCCCTCTGACCTTGTATTAC | Gain of 2093_Teak conKI allele |
| | | validation |

The targeting vector was entirely sequenced and then linearized by digestion with PmeI before electroporation into C57BL/6 Bruce4 ES cells (Kontgen et al., Targeted disruption of the MHC class II Aa gene in C57BL/6 mice, Int. Immunol. 1993; 5(8):957-64). Neo-resistant ES cell clones were screened by qPCR to identify potentially targeted clones.

Murine ES cells were targeted through homologous recombination. TaqMan® copy number reference assays were used to measure copy number in the genome. Two pairs of primers were used to amplify the WT locus at the extreme 5′ and 3′ positions to detect 2 copies from the WT allele and 1 copy from the targeted allele (primers, 2093_Lo5WT and 2093_LoWT3). Another primer pair targeting Neo sequence was used to test the targeted allele (primer, 1638_goNoz). Two genes from Y chromosome (1 copy) and chromosome 8 (2 copy) were used as control. Two positive clones, Clones I_1D08 and I_1G08, were confirmed as correctly targeted and were used for injection into goGermline blastocysts.

Mice heterozygous for a BiP-FLAG allele were produced as follows. ES cells from clones I_1D08 and I_1G08 were injected into goGermline donor blastocysts to generate chimeras. A total of 84 injected blastocysts were transferred into 7 recipient hosts. These resulted in 35 offspring, of which 28 were male chimeras. Four males were chosen for mating with homozygous Flp mice. A total of 17 pups was born from three litters, including 10 WT and 7 WT/conKI (FIG. 1B).

Primary hepatocytes and skin fibroblasts were isolated and cultured as follows. Mouse primary hepatocytes were isolated by portal vein perfusion of collagenase as described (Wang et al., IRE1alpha-XBP1s induces PDI expression to increase MTP activity for hepatic VLDL assembly and lipid homeostasis, Cell Metab. 2012; 16(4):473-86). Murine skin fibroblasts were prepared by collagenase (Type II and Type IV, Sigma) digestion of abdominal skins dissected from a female BiP-FLAG-Heterozygous (Het) mouse and an Hspa5 wild type littermate (6-weeks old). The primary hepatocytes and skin fibroblasts were cultured in DMEM/10% FBS. After overnight culture, cells were transduced with Ad-βGal or Ad-Cre at an MOI of 34. Where specified, cells were treated with castanospermine (CST) or tunicamycin (Tm) to induce ER stress.

Mouse experiments were performed as follows. Four female BiP-FLAG-Het mice and 4 of their female littermates were used for an in vivo experiment. They were infused with AAV8-TBG-Cre (2.5×10^11 vg/mouse) through tail vein injection at 6.5 weeks of age. After 10 days, mice were treated with Tm (1 mg/Kg) or vehicle (saline) through I.P. injection and were sacrificed for tissue collection after 17 h.

qRT-PCR and qPCR analyses were performed as follows. Total RNAs were extracted from isolated liver by RNeasy Mini Kit (Qiagen). cDNAs were synthesized by iScript cDNA Synthesis kit (Bio-Rad Laboratories, Inc). The relative mRNA levels were measured by qRT-PCR with iTaq Universal SYBR green Supermix (Bio-Rad Laboratories, Inc). All primers are listed in Table 1.

Immunofluorescence microscopy was performed as follows. Cells were plated on coverslips for overnight and fixed with 4% PFA. Cells and Sections were stained with the following antibodies; FLAG (M2, Sigma), α-PDIA6 (18233-1-AP, Proteintech), and DAPI (Fisher Scientific). For secondary antibodies we used: Alexa Fluor 488 goat α-rabbit IgG, Alexa Fluor 594 goat α-mouse IgG, anti-bodies (Invitrogen). Images were taken by a Zeiss LSM 710 confocal microscope with a 20× and 63× objective lenses. Scale bars are indicated in the figures.

All Western blots were performed separating proteins by SDS-PAGE on a 5-15% gradient polyacrylamide gel for transfer onto nitrocellulose membranes, followed by blocking with Licor Blocking solution and incubation with primary and fluorescent-labeled secondary antibodies (Licor). The immune-fluorescent signals were captured using a Licor scanner. The key primary antibodies used in this study were as follows: Flag (M2, Sigma), BiP(3177, CST), KDEL (SC-58774, SCBT), PDIA4 (14712-1-AP, Proteintech), PDIA6 (18233-1-AP, Proteintech), β-Actin (8H10D10, CST).

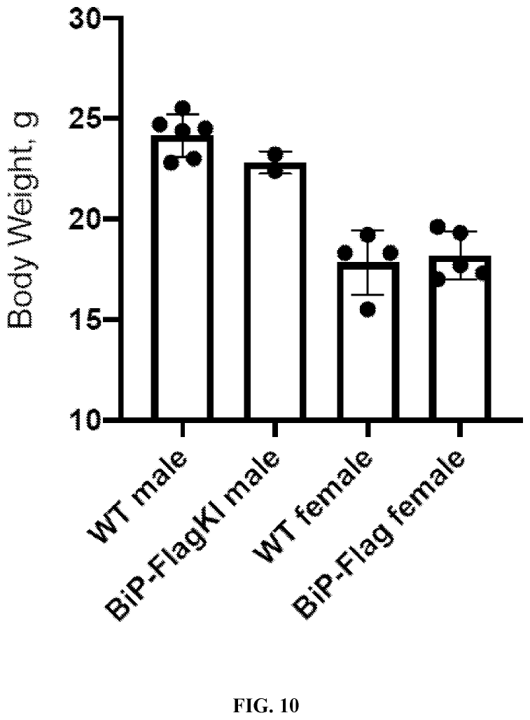

BiP-FLAG conditional knock-in mice were generated by targeting exon9 CDS region and flanking of with LoxP sites via gene targeting in Bruce4 C57BL/6 embryonic stem (ES) cells (Kontgen et al., Targeted disruption of the MHC class II Aa gene in C57BL/6 mice, Int. Immunol. 1993; 5(8):957-64). Gene-targeted ES cell clones were identified, and cells then injected into goGermline blastocysts (Koentgen et al., Exclusive transmission of the embryonic stem cell-derived genome through the mouse germline, Genesis 2016; 54(6):326-33; and Zhou et al., The testicular soma of Tsc22d3 knockout mice supports spermatogenesis and germline transmission from spermatogonial stem cell lines upon transplantation, Genesis 2019; 57(6):e23295). Male chimeric mice were bred with Flp female mice to delete the Neo cassette and establish heterozygous germline offspring on a C57BL/6 background (FIG. 1A). TaqMan® copy number assay was used to genotype the offspring (FIG. 1B). A total of 17 pups was born from three litters, including 10 WT and 7 WT/conKI (41% observed vs. 50% expected). All of these pups grew normally and appeared healthy. No difference in body weights between genotypes was observed (FIG. 10).

To test if the Cre-induced Hspa5-FLAG allele can deplete the endogenous Hspa5 allele, AAV-Cre was injected by intravenous injection into mouse tails as described for the in vivo experiments. To measure mRNA expression for the endogenous and targeted Hspa5 allele, qRT-PCR was performed with primers directed at the targeted region, including crossing FLAG region and within the FLAG region. With AAV-Cre induced LoxP deletion in liver, WT mice demonstrated an ˜2-fold increased expression compared to the BiP-FLAG-Het mice with primer Hspa5-exon 9 that identifies the WT allele. While the other Hspa5 primer that does not target the FLAG region did not show significant difference between the WT and knock-in mice. The primer targeting the FLAG sequence was only observed upon amplification in BiP-FLAG-Het mice. This confirmed the FLAG knock-in into the Hspa5 locus at exon 9. (FIG. 1C).

### Example 2

### BiP-FLAG Expression Ex Vivo in Primary Hepatocytes and Skin Fibroblasts Isolated from BiP-FLAG Heterozygous (Het) Mice

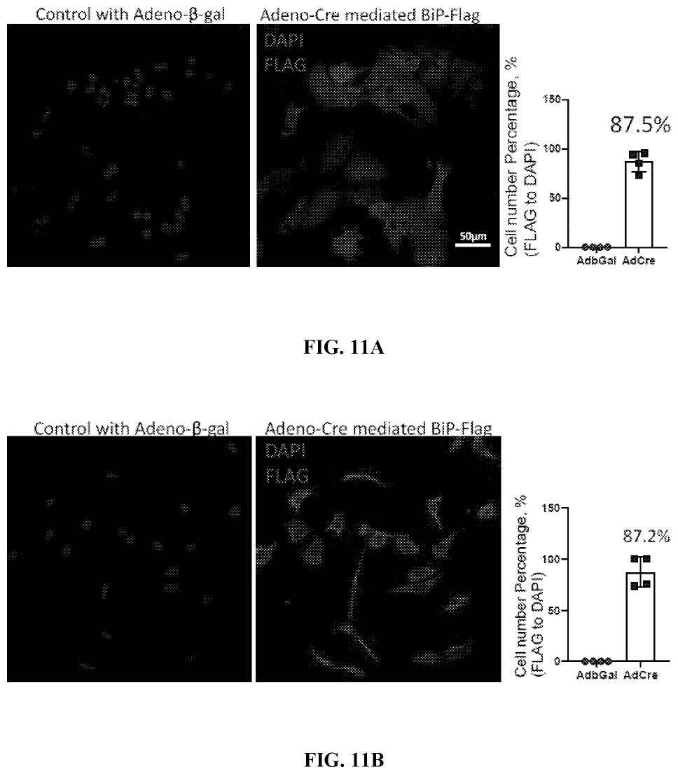

To activate BiP-FLAG expression ex vivo, primary hepatocytes and skin fibroblasts were isolated from a heterozygous BiP-FLAG mouse and transduced with Ad-Cre to induce Cre-mediated deletion of the floxed Hspa5 segment. At 24 h after Ad-Cre-transduction, approximately 90% of the BiP-FLAG-Het hepatocytes and fibroblasts were positive for FLAG immunofluorescence (FIG. 11).

In hepatocytes, western blot analysis detected BiP-FLAG migrating slightly above endogenous BiP in the BiP-FLAG-Het hepatocytes as early as 22 h after Ad-Cre transduction (data not shown). By 3 days after Ad-Cre transduction, the steady-state-level of BiP-FLAG was about 50% of the endogenous BiP under basal conditions but increased to a level similar to that of the endogenous BiP produced from the untargeted Hspa5 allele after Tm-treatment for 20 h, based on western blotting analysis with a rabbit anti-BiP monoclonal antibody (FIG. 2), suggesting that the 3xFLAG insertion into the BiP C-terminus does not alter Hspa5 expression in the mouse model. Significantly, very similar levels of total BiP were observed in the Ad-βGal- and Ad-Cre-transduced BiP-FLAG-Het hepatocytes after 20 h Tm-treatment (FIG. 2), suggesting that the genetic modification of Hspa5 did not alter the UPR response.

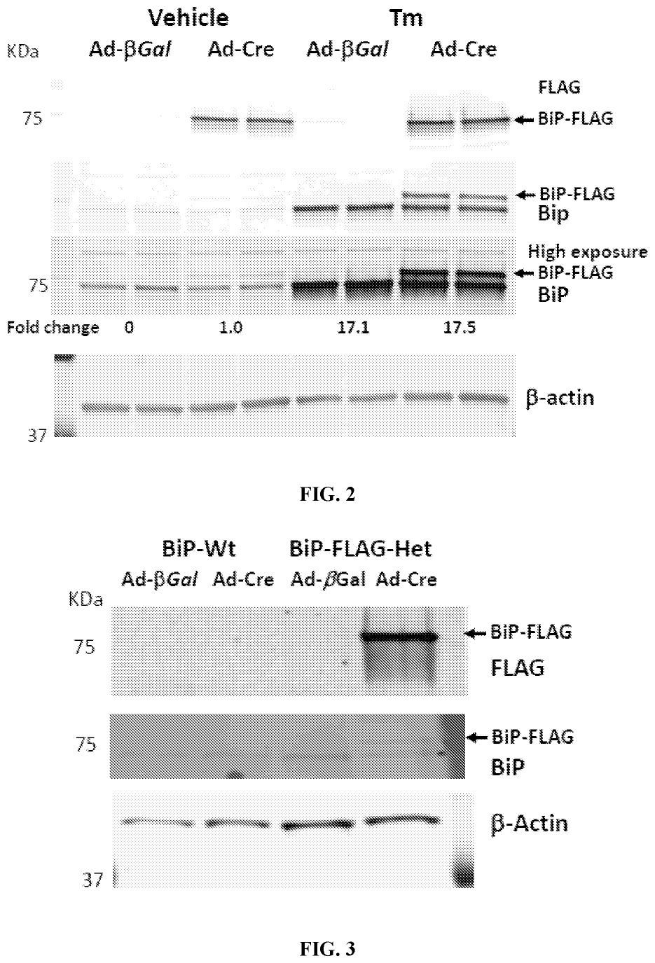

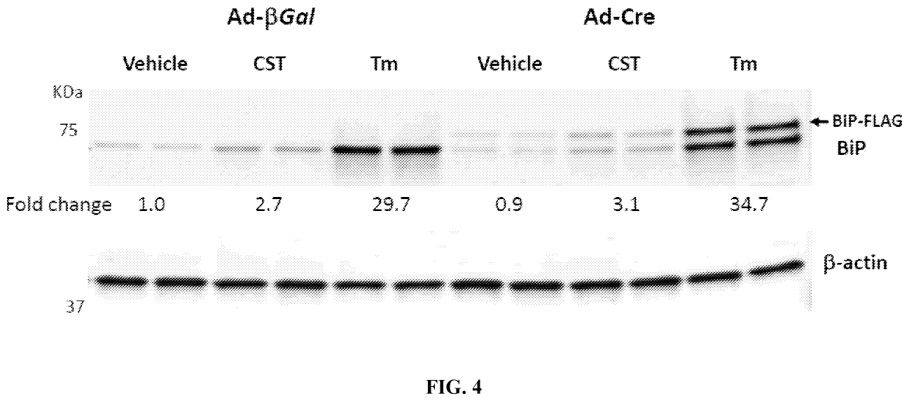

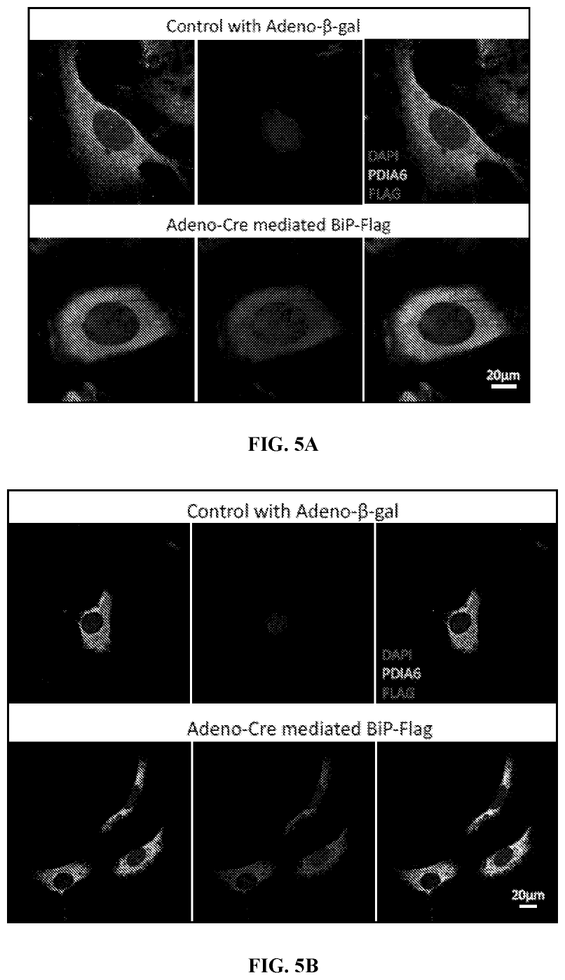

Unlike the primary hepatocytes, Ad-Cre activation of the BiP-FLAG knock-in locus in BiP-FLAG-Het primary skin fibroblasts resulted in equal levels of endogenous BiP and BiP-FLAG (FIG. 3). This difference between skin fibroblasts and primary hepatocytes may be explained by the fact that fibroblasts, but not hepatocytes, proliferate in vitro, leading to a dilution of the preexisting endogenous BiP in the fibroblasts. Significantly, the increase in endogenous BiP and BiP-FLAG were nearly identical in response to castanospermine (CST)- or Tm-treatment to activate the UPR (FIG. 4).

### Example 3

### BiP-FLAG is Localized to the ER

An essential question is whether tagging the C-terminus of BiP may alter its intracellular localization as the FLAG tag is adjacent to the KDEL ER retention signal. Immunofluorescence microscopy showed that BiP-FLAG colocalized with the ER localized PDIA6 both in Ad-Cre-transduced BiP-FLAG-Het primary hepatocytes (FIG. 5A) and skin fibroblasts (FIG. 5B), importantly demonstrating that insertion of the 3xFLAG tag into BiP did not alter its cellular localization.

### Example 4

### Hepatocyte-Specific Cre-Expression in BiP-FLAG-Het Mice Demonstrates Intact Functional Activities of BiP-FLAG In Vivo

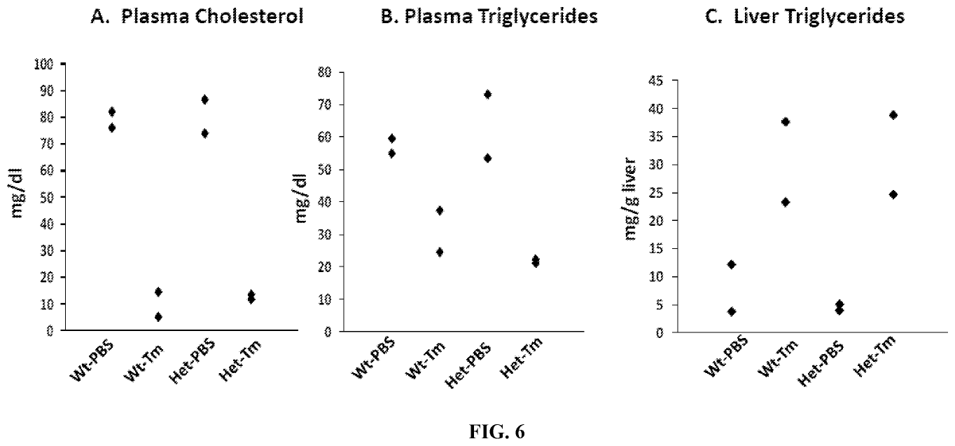

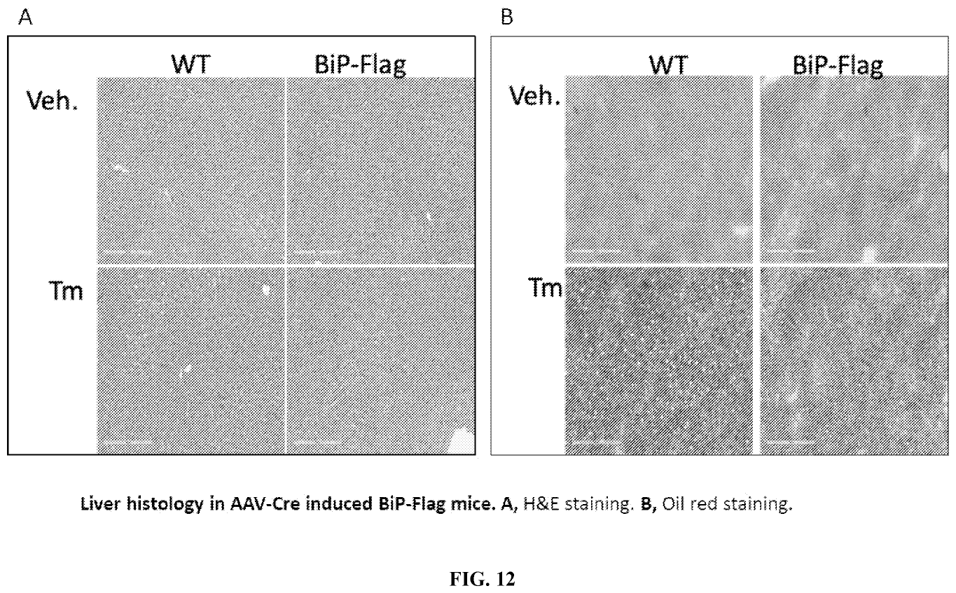

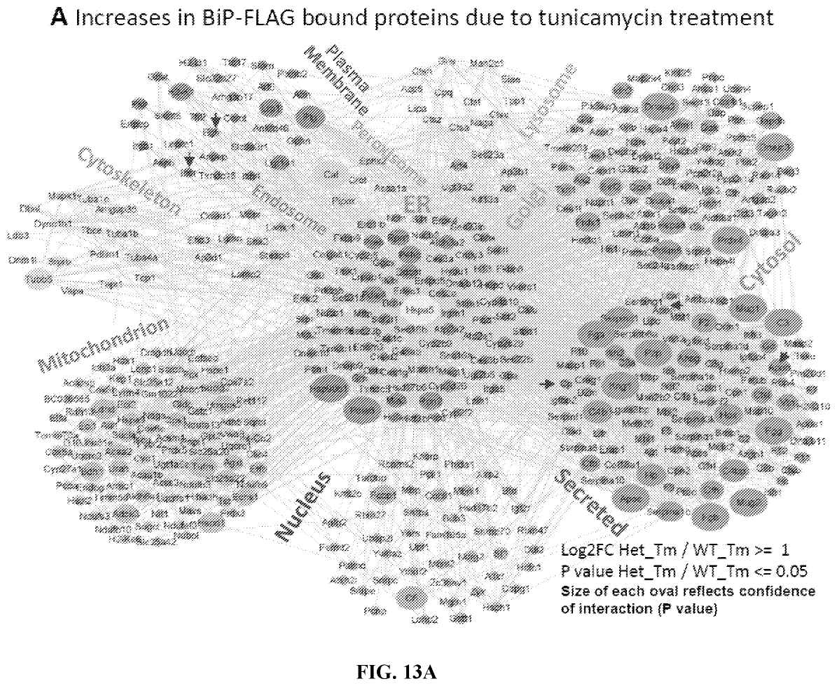

Together, the above findings show that BiP-FLAG knock-in did not alter the expression, localization or the functional activity of the endogenous or the modified Hspa5 alleles. To confirm these findings in vivo and to explore the feasibility for hepatocyte-specific BiP-FLAG knock-in, AAV8-TBG-Cre was infused into 4 BiP-FLAG-Het mice and 4 WT littermates to express Cre selectively in hepatocytes. The TBG promoter is a hybrid promoter comprised of the human thyroxine-binding globulin promoter and microglobin/bikunin enhancer that is specifically expressed in hepatocytes. These mice were treated with Tm (1 mg/Kg) or vehicle (saline) at day 10 after AAV8-infusion for 17 h. Cre-mediated activation of BiP-FLAG in hepatocytes of BiP-FLAG-Het mice did not alter plasma or hepatic lipid levels and did not alter liver morphology in the absence or presence of Tm-treatment (FIGS. 6A-C; FIG. 12). In addition, BiP-FLAG was detected in nearly all hepatocytes in the livers of both AAV8-TBG-Cre-infused BiP-FLAG-Het mice.

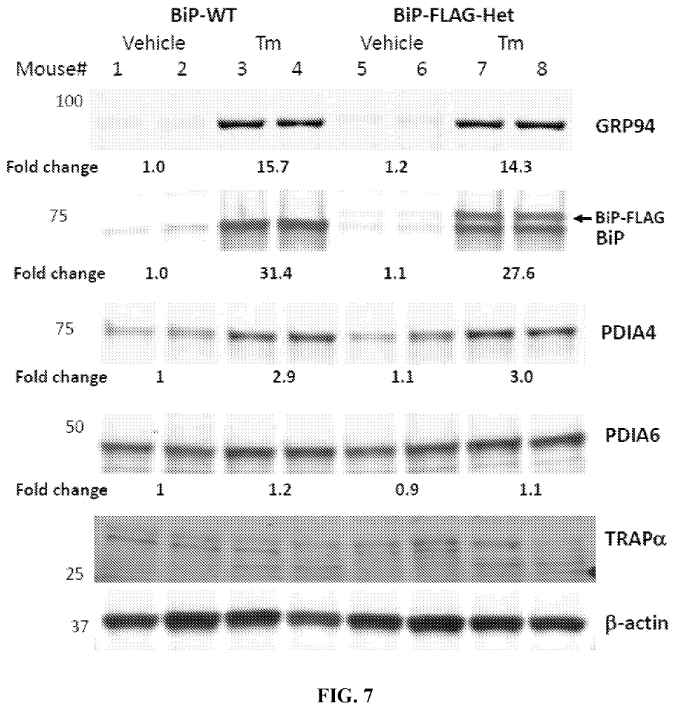

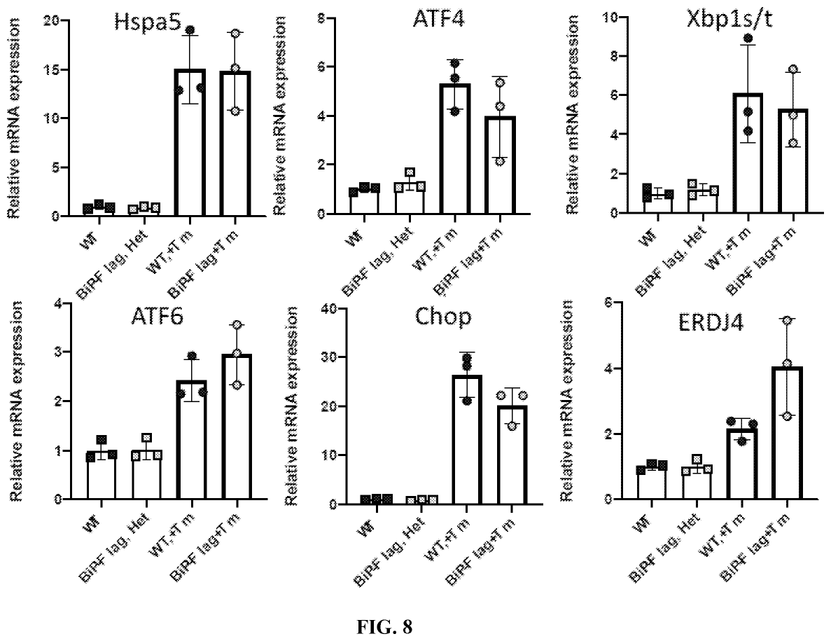

Like the Ad-Cre-transduced BiP-FLAG-Het skin fibroblasts, there were equivalent levels of endogenous BiP and BiP-FLAG in the livers of the AAV8-TBG-Cre-treated BiP-FLAG-Het mice with or without Tm-treatment (FIG. 7). Importantly, BiP-FLAG knock-in did not alter expression of GRP94, PDIA4 and PDIA6 as well as BiP in the livers under basal or Tm-induced conditions (FIG. 7). qRT-PCT assay demonstrated that activation of the Hspa5-FLAG allele did not alter expression of key UPR genes under basal or induced conditions (FIG. 8).

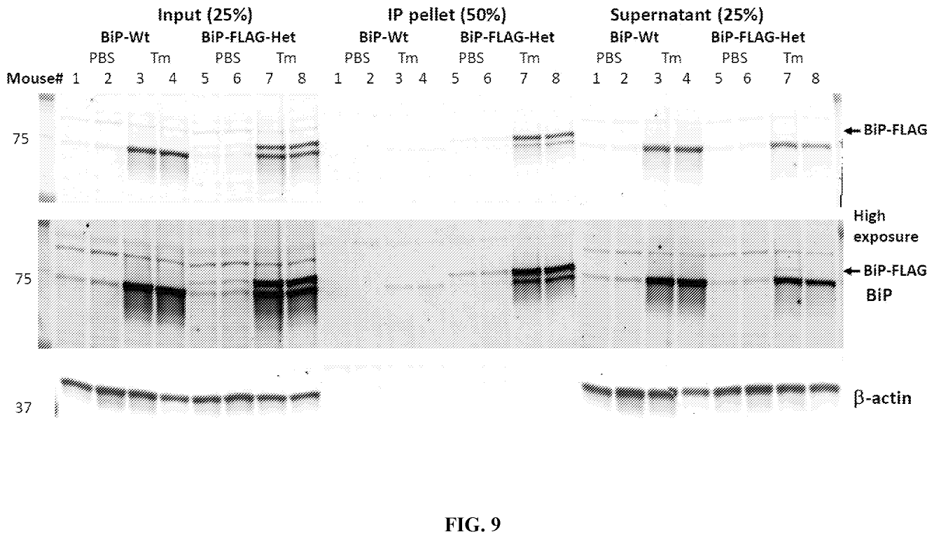

To confirm the ability of anti-FLAG antibody to immunoprecipitate (IP) BiP-FLAG synthesized in vivo, FLAG-IP assays of liver lysates prepared from the BiP-FLAG-Het mice were performed. A mouse anti-FLAG antibody completely depleted BiP-FLAG from the IP supernatants of the AAV8-TBG-Cre-treated BiP-FLAG-Het liver lysates (FIG. 9), demonstrating a high efficiency for BiP-FLAG pulldown. The finding that a significant amount of endogenous BiP was pulled down with BiP-FLAG from the liver lysates of the AAV8-TBG-infected BiP-FLAG-Het mice, especially those with Tm-treatment (FIG. 9), indicates that BiP-FLAG was pulled down as protein complexes.

### Example 5

### Affinity Purification-Mass Spectrometry (AP-MS) of BiP-FLAG Protein Complexes

To characterize the protein complexes that are pulled down with BiP-FLAG from lysates of the BiP-FLAG-expressing livers through FLAG-affinity purification, AP-MS analysis on livers of tunicamycin- or vehicle-treated BiP-FLAG heterozygous mice as described was carried out, with tunicamycin (Tm)- or vehicle (Veh)-treated wild type littermates as negative controls.

For this purpose, liver samples were lysed in a buffer containing 50 mM Hepes-NaOH pH 8.0, 100 mM KCl, 2 mM EDTA, 0.5% NP40 and 10% glycerol, supplemented with protease and phosphatase inhibitor cocktails. The liver lysates were centrifuged at 15,000 g for 20 min at 4 C. The resultant supernatants were subjected to immunoprecipitation with M2 anti-FLAG magnetic beads (Sigma). A sample of beads was removed for Western blot and the majority of beads were subjected to denaturation, reduction and overnight trypsin/lys-C mix digestion for 2D LC-MS/MS. MS/MS spectra were searched against the Mus musculus Uniprot protein sequence database using Maxquant (version 1.5.5.1) with false discovery rate (FDR) set to 1%. MSStats was used to calculate a confidence (p-value) and fold change of BiP-FLAG Het_Tm IP/BiP-FLAG Het_Veh after correction with Wt_Tm IP and Wt_Veh IP, respectively.

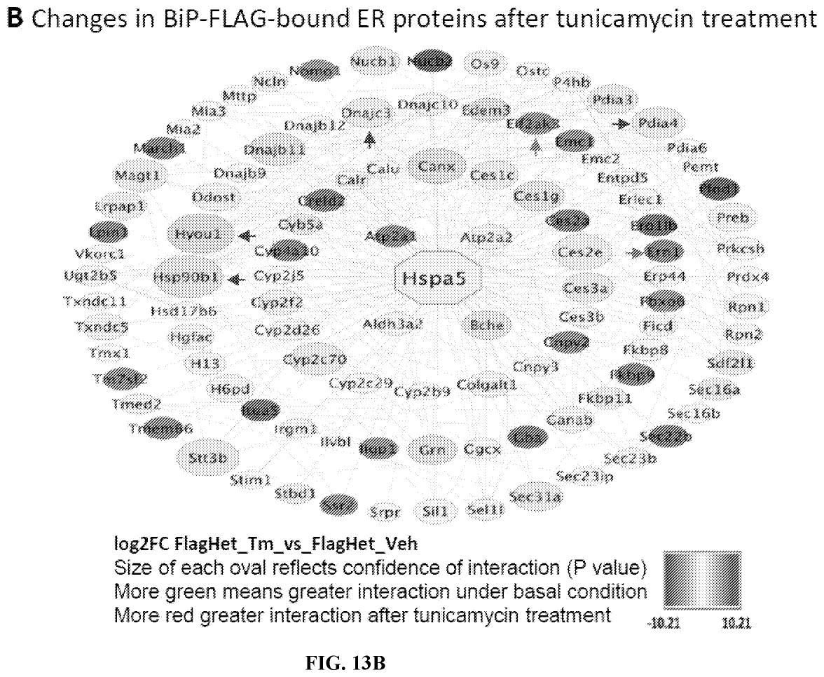

FIG. 13A shows all of the proteins that had increased interaction with BiP-FLAG in response to Tm treatment. Importantly, most of these proteins are N-glycosylated proteins, eg, insulin receptor (Insr) and EGF receptor (Egfr) in the plasma membrane and secretory proteins such as apolipoprotein B (Apob), apolipoprotein H (Apoh) and ceruloplasmin (Cp) (FIG. 10A, red arrows). Tm is a potent blocker of N-link glycosylation. Inhibition of N-glycosylation modification of the glycoproteins results in their misfolding in the ER. Thus, the increase in binding of these proteins to BiP-FLAG in the livers of the Tm-treated BiP-FLAG mice provides direct evidence that misfolded ER proteins are precipitated with BiP-FLAG with anti-FLAG magnetic beads as components of BiP-interactome.

FIG. 13B summarizes all of the ER proteins in the BiP-FLAG-expressing livers whose interactions with BiP-FLAG were affected by TM-treatment. Interestingly, the binding of PERK (Eif2ak3) and IRE1α (Ern1) to BiP-FLAG were greatly reduced after Tm-treatment (FIG. 13B, gray arrows). This is a very significant finding because: a) it for the first time, provides in vivo evidence that these two major UPR sensors bind to BiP under non-stressed condition and they are released from BiP upon UPR activation; and b) it further demonstrates the specificity of the proteins that are co-IP with BiP-FLAG in BiP-FLAG-expressing mouse livers. As expected, the interactions of several ER chaperone proteins with BiP-FLAG, including Grp94 (Hsp90b1), GRP170 (Hyou1), and P58 (Dnajc3), were significantly increased in the Tm-treated BiP-FLAG Het livers, providing further evidence of the integrity of the BiP-interactome purified through the usage of BiP-FLAG.

Together, these findings from AP-MS analysis of the BiP-FLAG complexes isolated from livers of our BiP-FLAG mice under basal condition and Tm-induced ER stress clearly demonstrate the feasibility of the unique in vivo model to detect changes in the BiP interaction network in response to ER stress and its usage to identify ER misfolded proteins.

What is claimed is:

1. A model of protein folding comprising:
a transgenic animal with a transgene comprising an epitope tag in the Hspa5 gene.

2. The model of claim 1, wherein the transgenic animal comprises a mammal.

3. The model of claim 1, wherein the transgenic animal is selected from the group consisting of: a mouse, a rat, and a monkey.

4. The model of claim 1, wherein the transgenic animal is a mouse.

5. The model of claim 1, wherein the transgenic animal is produced via homologous recombination.

6. The model of claim 1, wherein the epitope tag is selected from the group consisting of: GST, streptavidin, poly(His), FLAG-tag, V5-tag, Myc-tag, HA-tag, Spot-tag, T7-tag, and NE-tag.

7. The model of claim 1, wherein the epitope tag comprises a FLAG-tag.

8. The model of claim 7, wherein the FLAG-tag comprises at least three FLAG sequences.

9. A BiP-FLAG mouse characterized by FLAG-tagged BiP-client complexes.

10. The BiP-FLAG mouse of claim 9, wherein the FLAG-tagged BiP-client complexes comprise at least three FLAG sequences.
